SEQUENCE LISTING (11 sequences)

SEQ ID NO: 1 - DNA, 2031 bp, Nanoarchaeum equitans
  gene (1)..(2031): large fragment for Neq DNA polymerase
atgttacaccaactccccacgatggttgtagaagaaaaggcggtaaaagaggaagaagggtatagcgtgctaaaatgttattggattaatatagagaacacccctttagacgaggtaattttaataggtaaagacgaaaataatagagcttgtgaagttataattccatacaaatggtatttctattttgaaggcgatataaaggatttagaagaattcgctaacaacaaaaaaataaaaatcgaatatacaaaggagcaaaagaaatatatagaaaaaccaaaagatgtttataaagtatatgttttgcataaacattatccaatactaaaagaattcattaaagaaaagggctataaaaaatacgaaaccgatataaatgtttataggaagtttttaatagataaagggatagagccttttgaatggtttgaggtagaaggcaaaattttattatctacctctaacaaagttagaataaaagcacaaagtataaaaagattgtatgaaaagactaagccatcggttttagcttttgatatagaagtttacagtgaggctttccctaatcctgaaaaagacaaaataatatctatagccctttatggagacaattacgaaggggttatctcttacaaaggagaaccaactataaaagttaataccgaatatgaattaattgagaaatttgtcgaaataatagaaagcttaaaaccagacataatagttacatacaatggggataatttcgatatagactttttagtgaaaagggcttctttatacaatataaggctaccaataaaattggttaacaaaaaagagcctacttataattttagggaaagcgcacatgtagatttgtataaaacaattactaccatatataaaacccaattgtctacccaaacatattcattaaatgaagtagctaaagaaattcttggagaggagaaaatttatgattatgaaaacatgttatatgattgggccataggcaattataacaaagtgttcgaatacaatttaaaagatgccgaattaacatataagctattcaaatactatgaaaatgatttattggaattagcaagattggttaaccaaccattatttgatgtatctaggtttagctatagtaatatagttgaatggtatctaatcaaaaaaagcagaaaatataatgaaattgtgcctaacaaaccaaaaatggaagaagtagagagaagaaaattaaatacctatgcaggagcattcgtttacgaaccaaaacccggtttgtatgagaatttagctgtactggatttcgcttctctgtatccttcaattatattagagcataatgtttctccaggcacaatatattgtgagcatgatgattgtaaacaaaatggggtagaagcgataataaataatgagaaaaaatatgtgtggttttgcaaaaaagtaaaagggtttattccaacggtattagagcatttgtatacaaaaaggctagaacttaagagaaaactgaaagaactagatagggatagtgaagaatataaaattataaatgctaagcaagcagtattgaaaataataattaatgcaacctatggctatatgggtttcccaaatgcgagatggtattgcatagactgtgctgcggcagtagcagcttggggcaggaaatacattaattatatattaaaaagggccgaagaagaaggattcaaagtaatttatggagattctataatggatactgaaatagaggttatagaaaatggtataaaaaagaaagaaaagctaagcgatttgtttaacaaatactatgccggttttcaaataggggagaaacattatgctttccctccagatttgtatgtatatgatggagaaagatgggttaaagtatattcaataataaaacatgaaaccgagaccgatttatatgaaataaatgggataacattaagtgcaaaccatttagttttaagcaaaggcaattgggttaaagccaaggaatatgaaaataaaaataattaa

SEQ ID NO: 2 - protein, 676 aa, Nanoarchaeum equitans
  PEPTIDE (1)..(676): large fragment for Neq DNA polymerase
MLHQLPTMVVEEKAVKEEEGYSVLKCYWINIENTPLDEVILIGKDENNRACEVIIPYKWYFYFEGDIKDLEEFANNKKIKIEYTKEQKKYIEKPKDVYKVYVLHKHYPILKEFIKEKGYKKYETDINVYRKFLIDKGIEPFEWFEVEGKILLSTSNKVRIKAQSIKRLYEKTKPSVLAFDIEVYSEAFPNPEKDKIISIALYGDNYEGVISYKGEPTIKVNTEYELIEKFVEIIESLKPDIIVTYNGDNFDIDFLVKRASLYNIRLPIKLVNKKEPTYNFRESAHVDLYKTITTIYKTQLSTQTYSLNEVAKEILGEEKIYDYENMLYDWAIGNYNKVFEYNLKDAELTYKLFKYYENDLLELARLVNQPLFDVSRFSYSNIVEWYLIKKSRKYNEIVPNKPKMEEVERRKLNTYAGAFVYEPKPGLYENLAVLDFASLYPSIILEHNVSPGTIYCEHDDCKQNGVEAIINNEKKYVWFCKKVKGFIPTVLEHLYTKRLELKRKLKELDRDSEEYKIINAKQAVLKIIINATYGYMGFPNARWYCIDCAAAVAAWGRKYINYILKRAEEEGFKVIYGDSIMDTEIEVIENGIKKKEKLSDLFNKYYAGFQIGEKHYAFPPDLYVYDGERWVKVYSIIKHETETDLYEINGITLSANHLVLSKGNWVKAKEYENKNN

SEQ ID NO: 3 - DNA, 762 bp, Nanoarchaeum equitans
  gene (1)..(762): small fragment for Neq DNA polymerase
atgcgctatcttggcaaaaagagagttattttatatgatttatctactgaatcgggtaaattttatgttaatgggctagttttacacaataccgattcattattcatttctggggacaaagacaaagtattagaatttttagagaaagtaaataaagaattacccggtaaaatacaattagatttagaagatttctatgttagagggatattcgtaaaaaagaggggtgaacaaaagggggcaaaaaagaaatatgctttattaagcgaacaaggttacataaagctaaggggcttcgaagcagtaagaacagactgggctcccatagttaaagaagtccaaacaaagctattggaaattttgctaaaagaaggtaacatagaaaaagcaagacaatacataaaagaaattattagaaagctaagaaatagagaaataccatgggagaagcttttaattacagaaacgataagaaagcctttagaaaaatacaaagttgaagctcctcatgtggcagcagcaaaaaaatataaaaggttgggctataaagttatgcctggctttagagttagatatttagtggtaggtagcactggaagggtttcagatagaattaaaatagacaaagaagttaggggtaatgaatatgaccccgaatactacatagaaaaacaactattgcctgcagtagagcaaatattagaatctgtaggtattaaagacacattcacaggcaaaaaactaacagatttctttaaatga

SEQ ID NO: 4 - protein, 253 aa, Nanoarchaeum equitans
  PEPTIDE (1)..(253): small fragment for Neq DNA polymerase
MRYLGKKRVILYDLSTESGKFYVNGLVLHNTDSLFISGDKDKVLEFLEKVNKELPGKIQLDLEDFYVRGIFVKKRGEQKGAKKKYALLSEQGYIKLRGFEAVRTDWAPIVKEVQTKLLEILLKEGNIEKARQYIKEIIRKLRNREIPWEKLLITETIRKPLEKYKVEAPHVAAAKKYKRLGYKVMPGFRVRYLVVGSTGRVSDRIKIDKEVRGNEYDPEYYIEKQLLPAVEQILESVGIKDTFTGKKLTDFFK

SEQ ID NO: 5 - DNA, 2406 bp, Artificial Sequence
  active Neq DNA polymerase
atgttacaccaactccccacgatggttgtagaagaaaaggcggtaaaagaggaagaagggtatagcgtgctaaaatgttattggattaatatagagaacacccctttagacgaggtaattttaataggtaaagacgaaaataatagagcttgtgaagttataattccatacaaatggtatttctattttgaaggcgatataaaggatttagaagaattcgctaacaacaaaaaaataaaaatcgaatatacaaaggagcaaaagaaatatatagaaaaaccaaaagatgtttataaagtatatgttttgcataaacattatccaatactaaaagaattcattaaagaaaagggctataaaaaatacgaaaccgatataaatgtttataggaagtttttaatagataaagggatagagccttttgaatggtttgaggtagaaggcaaaattttattatctacctctaacaaagttagaataaaagcacaaagtataaaaagattgtatgaaaagactaagccatcggttttagcttttgatatagaagtttacagtgaggctttccctaatcctgaaaaagacaaaataatatctatagccctttatggagacaattacgaaggggttatctcttacaaaggagaaccaactataaaagttaataccgaatatgaattaattgagaaatttgtcgaaataatagaaagcttaaaaccagacataatagttacatacaatggggataatttcgatatagactttttagtgaaaagggcttctttatacaatataaggctaccaataaaattggttaacaaaaaagagcctacttataattttagggaaagcgcacatgtagatttgtataaaacaattactaccatatataaaacccaattgtctacccaaacatattcattaaatgaagtagctaaagaaattcttggagaggagaaaatttatgattatgaaaacatgttatatgattgggccataggcaattataacaaagtgttcgaatacaatttaaaagatgccgaattaacatataagctattcaaatactatgaaaatgatttattggaattagcaagattggttaaccaaccattatttgatgtatctaggtttagctatagtaatatagttgaatggtatctaatcaaaaaaagcagaaaatataatgaaattgtgcctaacaaaccaaaaatggaagaagtagagagaagaaaattaaatacctatgcaggagcattcgtttacgaaccaaaacccggtttgtatgagaatttagctgtactggatttcgcttctctgtatccttcaattatattagagcataatgtttctccaggcacaatatattgtgagcatgatgattgtaaacaaaatggggtagaagcgataataaataatgagaaaaaatatgtgtggttttgcaaaaaagtaaaagggtttattccaacggtattagagcatttgtatacaaaaaggctagaacttaagagaaaactgaaagaactagatagggatagtgaagaatataaaattataaatgctaagcaagcagtattgaaaataataattaatgcaacctatggctatatgggtttcccaaatgcgagatggtattgcatagactgtgctgcggcagtagcagcttggggcaggaaatacattaattatatattaaaaagggccgaagaagaaggattcaaagtaatttatggagataccgattcattattcatttctggggacaaagacaaagtattagaatttttagagaaagtaaataaagaattacccggtaaaatacaattagatttagaagatttctatgttagagggatattcgtaaaaaagaggggtgaacaaaagggggcaaaaaagaaatatgctttattaagcgaacaaggttacataaagctaaggggcttcgaagcagtaagaacagactgggctcccatagttaaagaagtccaaacaaagctattggaaattttgctaaaagaaggtaacatagaaaaagcaagacaatacataaaagaaattattagaaagctaagaaatagagaaataccatgggagaagcttttaattacagaaacgataagaaagcctttagaaaaatacaaagttgaagctcctcatgtggcagcagcaaaaaaatataaaaggttgggctataaagttatgcctggctttagagttagatatttagtggtaggtagcactggaagggtttcagatagaattaaaatagacaaagaagttaggggtaatgaatatgaccccgaatactacatagaaaaacaactattgcctgcagtagagcaaatattagaatctgtaggtattaaagacacattcacaggcaaaaaactaacagatttctttaaatga

SEQ ID NO: 6 - DNA, 20 bp, Artificial Sequence
  Primer
aataacgtcggcaactttgg

SEQ ID NO: 7 - DNA, 20 bp, Artificial Sequence
  Primer
gttacgccaccagtcatcct

SEQ ID NO: 8 - DNA, 20 bp, Artificial Sequence
  Primer
agagatggccacggctgctt

SEQ ID NO: 9 - DNA, 19 bp, Artificial Sequence
  Primer
attgcggtggacgatggag

SEQ ID NO: 10 - DNA, 22 bp, Artificial Sequence
  Primer
acatttgcttctgacacaactg

SEQ ID NO: 11 - DNA, 21 bp, Artificial Sequence
  Primer
aggcagaatccagatgctcaa

(19) United States
(12) Patent Application Publication
(10) Pub. No.: US 2012/0135472 A1
(43) Pub. Date: May 31, 2012

(54) HOT-START PCR BASED ON THE PROTEIN TRANS-SPLICING OF NANOARCHAEUM EQUITANS DNA POLYMERASE

(75) Inventors: Suk Tae KWON, Suwon-si (KR); Sung Suk Cho, Suwon-si (KR); Jae Geun Song, Seoul (KR); In Hye Kim, Cheongju-si (KR); Kang Keun Lee, Suwon-si (KR); Man-Hui Youn, Incheon (KR)

(73) Assignee: RESEARCH & BUSINESS FOUNDATION SUNGKYUNKWAN UNIVERSITY, Suwon-si Gyeonggi-do (KR)

(21) Appl. No.: 13/306,915

(22) Filed: Nov. 29, 2011

(30) Foreign Application Priority Data
Nov. 29, 2010 (KR) 10-2010-0119895

(51) Int. Cl. C12P 19/34

(52) U.S. Cl. 435/91.2

(57) ABSTRACT

Disclosed is a hot-start PCR method, based on protein trans-splicing of intein-inserted large (Neq L) and small (Neq S) fragments of Neq DNA polymerase. The method comprises: preparing a PCR reaction mixture containing a sample DNA and primers; adding the Neq L fragment and the Neq S fragment together to the PCR reaction mixture, said Neq L fragment consisting of an amino acid sequence of SEQ ID NO: 2, with an intein amino acid sequence stretching from position 579 to 676 therein, said Neq S fragment consisting of an amino acid sequence of SEQ ID NO: 4 with an intein amino acid sequence stretching from position 1 to 30 therein; inducing the Neq L fragment and the Neq S fragment to undergo a protein trans-splicing process to form a polypeptide exhibiting Neq DNA polymerase activity; and performing a certain number of cycles of DNA denaturation, primer annealing and DNA extension.

### CROSS-REFERENCE(S) TO RELATED APPLICATIONS

The present application claims priority of Korean Patent Application No. 10-2010-0119895, filed on Nov. 29, 2010, which is incorporated herein by reference in its entirety.

### BACKGROUND OF THE INVENTION

1. Field of the Invention

The present invention relates to a novel hot-start PCR method based on the protein trans-splicing of Nanoarchaeum equitans DNA polymerase.

2. Description of the Related Art

A DNA polymerase (deoxyribonucleic acid polymerase, E.C. number 2.7.7.7) is an enzyme that synthesizes complementary DNA in the 5′→3′ direction on template DNA, and plays the most important role in DNA replication and repair.

Based on amino acid homology, DNA polymerases can be divided into at least six different families (family A, B, C, D, X and Y) (Ohmori, H. et al., 2001, Mol. Cell. 8: 7-8). Most DNA polymerases that belongs to family B can perform replication with high fidelity because of their 3′→5′ exonuclease activity, known as proofreading activity. The development of a PCR technique using thermostable DNA polymerase (Saiki, R. K. et al., 1988, Science 239, 487-491) has brought considerable interest to thermostable DNA polymerases, and these enzymes are being developed competitively from several thermophiles and hyperthermophiles. In particular, thermostable DNA polymerases from hyperthermophilic archaeons, such as Thermococcus litoralis and Pyrococcus furiosus, have been used in PCR requiring high fidelity because they have 3′→5′ exonuclease activity, known as proofreading activity, as well as DNA polymerization activity (Mattila, P. et al., 1991, Nucleic Acids Res. 19, 4967-4973; Lundberg, K. S. et al., 1991, Gene 108, 1-6).

An intein is a protein insertion sequence that is embedded in-frame within a precursor protein sequence. Intein sequences are removed from the precursor protein by a self-splicing process, and thus do not affect the structure and activity of the final protein made from the precursor protein (Perler, F. B. et al., 1994, Nucleic Acids Res. 22, 1125-1127).

Protein splicing is a post-translational processing event in which the intein is precisely self-excised from a precursor protein with the concomitant ligation of the flanking protein sequences, exteins, through a normal peptide bond (Kane, P. M. et al., 1990, Science 250, 651-657).

Nanoarchaeum equitans (Neq) is a nano-sized, hyperthermophilic anaerobe which was isolated from a submarine hot vent at the Kolbeinsey ridge in Iceland (Huber, H. et al., 2002, Nature 417, 63-67). This strain grows on the surface of a specific host, Ignicoccus sp. strain KIN4/I, under strictly anaerobic conditions.

Neq DNA polymerase is encoded by two genes, which are separated by 83,295 bp on the chromosome and individually contain an extein sequence and a split mini-intein encoding sequence (Waters, E. et al., 2003, Proc. Natl. Acad. Sci. USA 100, 12984-12988). Neq DNA polymerase is produced by genes which code for a large fragment (Neq L) and a small fragment (Neq S), respectively. That is, after being expressed from each genes, which are separate on the chromosome, respective polypeptides are linked by a peptide bond to form a single protein through protein trans-splicing, thereby yielding an active DNA polymerase (Choi J. J. et al., 2006, J. Mol. Biol. 356:1093-106).

The large fragment of Neq DNA polymerase consists of an extein region of 578 amino acid residues, which corresponds to the amino-terminal part (N-terminal part) of Neq DNA polymerase, and an intein region of 98 amino acid residues, which corresponds to the N-terminal part of a split mini-intein participating in protein trans-splicing. As for the small fragment of Neq DNA polymerase, it consists of an intein region of 30 amino acid residues, which corresponds to the carboxyl-terminal part (C-terminal part) of the split mini-intein, and an extein region of 223 amino acid residues, which corresponds to the C-terminal part of Neq DNA polymerase (Choi J. J. et al., 2006, J. Mol. Biol. 356:1093-106; Korean Patent No. 10-0793007 and U.S. Pat. No. 7,749,732).

After the construction of recombinant expression vectors carrying their respective genes, Neq L and Neq S were expressed in E. coli and purified. When these large and small fragments are incubated together at a high temperature, protein trans-splicing occurred between Neq L and Neq S (Choi J. J. et al., 2006, J. Mol. Biol. 356:1093-106; Korean Patent No. 10-0793007 and U.S. Pat. No. 7,749,732). Nowhere has the separate use of Neq L and Neq S in PCR been reported in previous documentation.

E. coli transformed with one expression vector carrying both the large and the small fragment of Neq DNA polymerase was incubated and ruptured by ultrasonification, after which the cell lysate was incubated at a high temperature to induce protein trans-splicing. As a result, a protein in which inteins were removed from Neq L and Neq S through protein trans-splicing and only exteins were linked by a peptide bond and it was designated Neq C (protein trans-spliced form of Neq DNA polymerase). Also, an extein-encoding region of the Neq DNA polymerase large fragment gene, from which an intein-encoding region is removed, is recombined with an extein-encoding region of the Neq DNA polymerase small fragment gene, from which an intein-encoding region is removed, to express as a single polypeptide chain. The expressed DNA polymerase is designated Neq P (genetically protein splicing-processed form of Neq DNA polymerase). It was found that Neq C and Neq P, prepared by different methods, are enzymes exhibiting the same activity and biochemical properties (Choi J. J. et al., 2006, J. Mol. Biol. 356:1093-106; Korean Patent No. 10-0793007 and U.S. Pat. No. 7,749,732).

The N-terminal domain of Archaeal family-B DNA polymerases contains a specialized pocket that binds to deaminated bases such as uracil and hypoxanthine to stop DNA replication (Fogg M. J. et al., 2002, Nat. Struct. Biol. 9: 922-927; Gill S. et al., 2007, J. Mol. Biol. 372: 855-863). The structure of Neq DNA polymerase is different from that of other family-B DNA polymerases. However, Neq DNA polymerase is an Archaeal family-B DNA polymerase that has no uracil-binding pockets so can successfully utilize deaminated bases. Recently, a preparation method of Neq plus DNA polymerase, which is a combination of Neq DNA polymerase and Taq DNA polymerase, and the PCR application of uracil-DNA glycosylase and dUTP have been reported (Choi J. J. et al., 2008, Appl. Environ. Microbio. 74: 6563-6569).

PCR (Polymerase Chain Reaction) is a molecular biological technique that is very usefully applied to identifying and determining the presence of an infection caused by virus or pathogenic bacteria. During various PCR procedures including sample selection, nucleic acid separation, sample transfer, PCR amplification, storage, post-electrophoresis recovery, etc., however, carry-over contamination may occur. Such carry-over contamination can bring about a false positive, giving rise to a decrease in the accuracy of clinical diagnosis if the contaminant, even in a trace amount, is amplified together with the target sample.

In order to prevent the occurrence of carry-over contamination in PCR, PCR employing dUTP instead of dTTP was suggested (Longo, M. C. et al, 1990, Gene, 93:125-128). There is also disclosed a PCR method in which template DNA is treated with UDG to remove DNA contaminated with a trace amount of uracil DNA and then heated to inactivate UDG, followed by performing PCR in the presence of dUTP instead of dTTP (Rys, P. N., and D. H. Persing. 1993. J. Clin. Microbiol. 31:2356-2360). As a result, commercially available PCR kit products are designed to encompass treatment with UDG in the PCR procedure.

Currently, one of the most interesting techniques in the PCR-related business is hot-start PCR. In typical PCR, non-specific primer binding takes place during the initial set up stages of the PCR such as when PCR components are mixed or upon initial PCR denaturation. Thus, the activity of the polymerase results in undesired PCR products, which compete with a PCR product of interest for subsequence amplification. It results in interference of the detection of the PCR product of interest. This non-specific amplification becomes a significant barrier particularly to the detection of target DNA present in a low number of copies, the amplification of low concentration DNA samples, and the performance of multiplex PCR using various primers at the same time. Hot-start PCR, which aims to reduce the amplification of undesired PCR products attributable to non-specific primer binding during the initial set up stages of PCR, is designed to allow the primer binding only at an elevated initial temperature so as to increase the specificity of the PCR product.

To date, hot-start PCR can be used for identification of infectious disease (e.g., HIV), amplification of DNA with low purity, real-time PCR, one-step RT-PCR, etc. Various aspects of related enzymes have also been studied.

At first, hot-start PCR was manually conducted. In this regard, one of the components necessary for PCR (for example, MgCl₂, Taq DNA polymerase, dNTP, and so on) is added only after the reaction mixture is heated to the denaturation temperature. This manual method is difficult to apply to the treatment of a large number of samples. Developed thereafter was a method which employs a solid wax-barrier between important PCR components. This wax-barrier melts only above elevated temperatures, so that all of the reaction components are mixed only at high temperature, preventing mis-primer binding and extension of mis-primed oligonucleotides (Chou Q. et al, 1992, Nucleic Acids Res. 20: 1717-1723). However, the wax-mediated hot start procedure suffers from the disadvantage of carrying a higher risk of contamination and being less convenient, because of the increased time required by sample processing, because of the solid wax-barrier that forms above the reaction mixture after finishing PCR, and due to increased PCR volume. To avoid these problems, the most commercially successful method has been to utilize a specific antibody for Taq DNA polymerase in PCR (Kellogg D. E. et al, 1994, Biotechniques 16: 1134-1137). The antibody-mediated PCR method has been adopted by some companies such as Invitrogen, and in this method DNA polymerase that is complexed with a specific antibody therefore exhibits no oligonucleotide extension activity at room temperature, but at elevated temperatures, the antibody dissociates from the complex, thus releasing the DNA polymerase, which can then function in DNA synthesis during PCR. Because the primers can be accurately bound to a target DNA at the elevated temperatures, only the target DNA can be specifically amplified. However, this method requires an excess of antibodies, which are generally expensive.

Another method of reducing non-specific amplification products involves the use of a chemically modified DNA polymerase. This technique was developed separately by Roche (Birch D. E. et al, 1996, Nature 381: 445-446, Birch D. E. et al., 1997, U.S. Pat. No. 5,677,152) and Qiagen (Ivanov Igor et al., 2001, U.S. Pat. No. 6,183,998) and has a hold on approximately 68% of the hot-start PCR market in U.S. In this method, the Taq DNA polymerase is inactive due to chemical modification and becomes active only after the initial incubation of the DNA polymerase for a certain period of time at elevated temperatures (e.g., 95° C., 10 min), thus preventing the production of non-specific DNA synthesis products during reaction set-up and the initial heating step of PCR. This method is also disadvantageous in that only approximately 30% of the enzyme is reactivated in the initial incubation, and it is not applicable to the amplification of long sequences due to the depurination of the template DNA upon reactivation at elevated temperatures. In spite of these drawbacks, the chemically modified enzymes are now used across a wide spectrum of types of PCR because of user convenience and high specificity. Other methods include the design of special primers (high cost for primer synthesis), magnesium precipitation (Barbesnes W M and Rowlyk K R, Molecular and Cellular Probes 16: 167-171) (a high concentration of Mg used, but an accurate concentration is impossible to determine), the use of pyrophosphatase and pyrophosphate (Bioneer, Korean Patent Application No. 10-2007-01090055) (instability of PCR master mixture). All of the methods above have the problems.

There is therefore a need for a PCR technique that can be performed simply and effectively at low cost. Based on the fact that protein trans-splicing takes place in Neq L and Neq S at high temperature, both of which contain inteins, the present inventors applied Neq L and Neq S design a novel hot-start PCR method in which no DNA polymerase activity appears at low temperatures due to the lack of trans-splicing whereas at high temperatures, trans-splicing occurs to remove the inteins with the concomitant linkage of the exteins, thus releasing active Neq DNA polymerase.

### SUMMARY OF THE INVENTION

It is an object of the present invention to provide a hot-start PCR method based on the protein trans-splicing of both the large and the small fragment of Neq DNA polymerase, which contain respective inteins therein.

It is another object of the present invention to provide a hot-start PCR method optimized by the protein trans-splicing of both Neq L and Neq S.

The present invention is based on the principle that when the large fragment (Neq L) and the small fragment (Neq S) of Neq DNA polymerase, both containing respective inteins therein, are heated together, protein trans-splicing occurs to remove the inteins with the concomitant linkage of flanking exteins, thereby releasing Neq DNA polymerase. The present invention is the first application to PCR with separate Neq L and Neq S.

In detail, the present invention contemplates a hot-start PCR method based on the protein trans-splicing of Neq L and Neq S, comprising:

preparing a PCR reaction mixture containing a sample DNA and primers;

adding Neq L and Neq S to the PCR reaction mixture, said Neq L consisting of the amino acid sequence of SEQ ID NO: 2, with an intein amino acid sequence stretching from position 579 to 676 therein, said Neq S consisting of the amino acid sequence of SEQ ID NO: 4 with an intein amino acid sequence stretching from position 1 to 30 therein;

inducing the Neq L and the Neq S to undergo a protein trans-splicing process to form a polypeptide (SEQ ID NO: 5) exhibiting Neq DNA polymerase activity; and

performing a certain number of cycles of DNA denaturation, primer annealing and DNA extension.

Nanoarchaeum equitans DNA polymerase (Neq DNA polymerase) comprises a large fragment (Neq L) and a small fragment (Neq S), which are separate from each other. Neq L consists of an extein region of 578 amino acid residues, which corresponds to the amino-terminal part of Neq DNA polymerase, and a mini intein region of 98 amino acid residues (SEQ ID NOS: 1 and 2). Neq S consists of an intein region of 30 amino acid residues and an extein region of 223 amino acid residues, which corresponds to the carboxyl terminal part of Neq DNA polymerase (SEQ ID NOS: 3 and 4).

Based on the finding that the inteins are excised from Neq L and Neq S by self-protein trans-splicing at elevated temperatures, with the concomitant construction of complete Neq DNA polymerase, inventors firstly used Neq L and Neq S, both containing respective inteins therein, for a hot-start PCR method in the present invention.

The hot-start PCR method of the present invention is characterized by inducing Neq L and Neq S to undergo protein trans-splicing at elevated temperatures before template denaturation. Subsequent to the production of a polypeptide exhibiting Neq DNA polymerase activity, any PCR steps, including DNA denaturation, primer annealing and DNA extension may be employed without limitation if they are well known in the art.

In addition to the DNA sample of interest, the primers complimentarily binding to the DNA and intein-inserted Neq L and Neq S, the PCR reaction mixture may contain any component necessary for PCR without limitation if it is well known in the art. Examples of the components containable in the PCR reaction mixture include PCR buffer (20 mM Tris-HCl (pH 8.0), 50 mM KCl, 1.5 mM MgCl₂, 0.01% BSA), dNTPs, RNase and sterile water, but are not limited thereto.

Although intein-inserted Neq L and Neq S are added to a PCR reaction mixture, protein trans-splicing does not occur at low temperature, so that DNA polymerization does not occur under the condition of the non-specific association of primers with a DNA template at low temperature.

In the hot-start PCR method of the present invention, protein trans-splicing occurs only at elevated temperatures, which allows the production of Neq DNA polymerase. The Neq DNA polymerase, in turn, participates in the amplification of DNA from the specific primer binding to the DNA template at elevated temperatures.

The polypeptide exhibiting Neq DNA polymerase activity may be produced after the trans-splicing of both Neq L and Neq S at 60˜95° C. for 2˜9 min.

In the present invention, normal Neq DNA polymerase was found to be produced only at high temperatures. In this regard, after intein-inserted Neq L and Neq S were added in the same molar quantities to the PCR reaction mixture, protein trans-splicing effects were analyzed versus reaction temperature and time by comparing the activity of the obtained DNA polymerases.

In the present invention, the production of normal Neq DNA polymerase was also confirmed by analyzing the effect of PCR conditions such as PCR cycles in PCR instrument on the protein trans-splicing of Neq L and Neq S and comparing the activity of obtained DNA polymerases.

Also, a determination was made concerning the levels of intein-inserted Neq L and Neq S in the PCR reaction mixture which provide the optimal amplification conditions for hot-start PCR. In detail, the PCR reaction mixture may contain both Neq L and Neq S in an amount of 0.2˜0.9 pmoles, with a molar ratio of Neq L:Neq S ranging from 1:1 to 1:1.6.

The PCR may be performed in the presence of dUTP (2′-deoxyuridine 5′-triphosphate).

Neq L and Neq S are used together with the PCR reaction mixture in a PCR kit. The PCR kit according to the present invention may comprise Neq L, Neq S, a vessel containing detection primers, a tube or container for amplification reaction, PCR buffer, dNTPs, RNase and sterile water.

The PCR kit of the present invention comprising Neq L and Neq S may be more effective than Taq DNA polymerase in applications in various fields including genetic engineering, molecular biology experiments, clinical diagnosis, forensic studies, etc.

Based on the protein trans-splicing of Neq L and Neq S, the hot-start PCR method of the present invention guarantees DNA amplification of high accuracy and specificity. Further, the hot-start PCR method can utilize uracil-DNA glycosylase (hereinafter referred to as “UDG”) in combination with dUTP in PCR for preventing carry-over contamination at high efficiency, compared to conventional DNA polymerases. Particularly in the presence of dUTP, the DNA polymerase of the present invention exhibits more specific amplification than Taq DNA polymerase, so that the hot-start PCR method can be effective at diagnosing diseases using real-time PCR employing UDG and dUTP.

### BRIEF DESCRIPTION OF THE DRAWINGS

The above and other objects, features and further advantages of the present invention will be more clearly understood from the following detailed description taken in conjunction with the accompanying drawings, in which:

FIG. 1A is a schematic gene map of the recombinant expression vector pENPL constructed by cloning Neq L gene to the expression vector pET-20b(+). FIG. 1B is a schematic gene map of the recombinant expression vector pENPS constructed by cloning an Neq S gene to the expression vector pET-22b(+). FIG. 1C shows amino acid sequences of mini inteins which respectively correspond to the carboxyl terminal portion of Neq L fragment and the amino terminal portion of Neq S fragment, both playing an important role in the protein trans-splicing of Neq DNA polymerase;

FIG. 2A shows protein trans-splicing effects versus reaction temperature and time when each of the Neq L and the Neq S fragments is used in an amount of 20 pmoles. Incubation was performed in a temperature range of from 50 to 95° C. for 1, 5 and 10 min, followed by analysis with SDS denaturation gel electrophoresis. FIG. 2B shows the activity of DNA polymerase of the protein trans-splicing reaction solution containing Neq L and Neq S versus reaction temperature and time;

FIG. 3A shows the effects of the pre-denaturation state and the number of PCR cycles on the protein trans-splicing of Neq L and Neq S as analyzed by SDS denaturation gel electrophoresis. The pre-denaturation was carried out at 95° C. for 0, 1 and 3 min and PCR was performed with 1, 2, 3, 4, 5, 10, 20 and 30 cycles. FIG. 3B shows the activity of DNA polymerase in the protein trans-splicing reaction solution containing Neq L and Neq S versus the number of PCR cycles;

FIG. 4 shows PCR products obtained using 0.1˜1.8 pmoles of an equimolar mixture of Neq L and Neq S in 20 μL of a PCR reaction mixture as analyzed by agarose gel electrophoresis;

FIG. 5 shows effects of the molar ratio between Neq L and Neq S on PCR amplification as measured by agarose gel electrophoresis. PCR was performed using 0.6 pmoles of Neq L in 20 μL of PCR reaction mixture containing 0.6˜0.96 pmoles of Neq S. 1 kb marker was run on lane M, Neq L 0.6 pmole/Neq S 0.6 pmole (1:1) on lane 1, Neq L 0.6 pmole/Neq S 0.72 pmole (1:1.2) on lane 2, Neq L 0.6 pmole/Neq S 0.84 pmole (1:1.4) on lane 3, and Neq L 0.6 pmole/Neq S 0.96 pmole (1:1.6) on lane 4; and

FIG. 6 shows PCR products obtained after performing PCR on human genomic DNA in the presence of Neq HS DNA polymerase (Neq DNA polymerase produced by combining Neq L and Neq S fragments), Taq DNA polymerase and Pfu DNA polymerase using (A) dNTP with a pair of primers targeting a β-actin gene (653 bp), (B) dUTP with a pair of primers targeting a ρ-actin gene (653 bp), (C) dNTP with a pair of primers targeting a hemoglobin gene (1581 bp) and (D) dUTP with a pair of primers targeting a hemoglobin gene (1581 bp).

### DESCRIPTION OF THE PREFERRED EMBODIMENTS

In the present invention, the amplification of target DNA by intein-inserted Neq L and Neq S was found to take place at higher efficiency than when conventional DNA polymerases were used, as determined by hot-start PCR in which a beta-actin gene and a hemoglobin gene were separately amplified with human genomic DNA in the presence of dNTP and dUTP.

To implement embodiments of the present invention, pure intein-inserted Neq L and Neq S must be easily obtained. Neq L and Neq S may be isolated and purified by the previously reported method (Choi J. J. et al., 2006, J. Mol. Biol. 356:1093-106; Korean Patent No. 10-0793007 and U.S. Pat. No. 7,749,732). Following purification, however, the dialysis of Neq S was carried out in a different manner. Neq S is purified with urea because it is expressed as an insoluble protein. When the urea was removed using a conventional dialysis method, the Neq S protein is apt to form precipitates, with the loss rate reaching as high as 90%. In contrast, when the Neq S protein solubilized with 8 M urea was refolded in a dialysis bag by gradient removal of urea and by gradient addition of a stabilizer, for example, a dialysis buffer containing 40% glycerol, as suggested by Kohyama et al., no precipitation occurred (Kohyama K. et al., 2009, J. Biochem. 147: 427-431).

The Neq L and the Neq S fragments according to the present invention can be expressed in E. coli using a conventional method. Also, the Neq L protein can be purified in a conventional manner. Because it is expressed in an insoluble form, however, the Neq S protein is solubilized with urea. To obtain the Neq S protein without loss, it may be refolded by gradient removal of urea and by gradient addition of 40% glycerol according to the Kohyama method.

The hot-start PCR polymerase consisting of an optimal mixture of Neq L and Neq S in accordance with the present invention exhibits more accurate PCR specificity on the human genome than does Taq DNA polymerase or Pfu DNA polymerase. In addition, the DNA polymerase consisting of an optimal mixture of Neq L and Neq S fragments can be used for PCR in the presence of dUTP. Particularly in the presence of dUTP, the polymerase of the present invention can perform PCR with higher amplification specificity within a shorter period of time, compared to Taq DNA polymerase.

That is, the DNA polymerase of the present invention can amplify only the target fragment at higher efficiency in the presence of dUTP. Exhibiting higher polymerization activity and amplification specificity in the presence of dUTP, compared to conventional polymerases (e.g., Taq DNA polymerase), the DNA polymerase consisting of an optimal mixture of Neq L and Neq S fragments in accordance with the present invention is highly suitable for use in PCR which is performed using UDG and dUTP to diagnose diseases.

A better understanding of the present invention may be obtained through the following examples which are set forth to illustrate, but are not to be construed as limiting the present invention.

### Example 1

### Expression and Purification of Neq L and Neq S

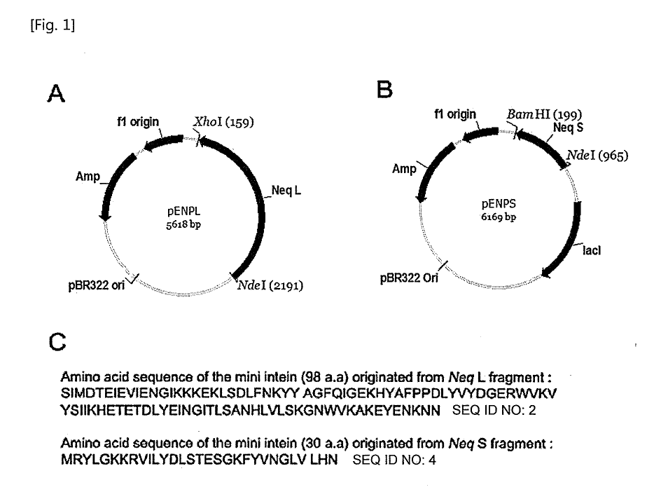

The Neq L fragment was obtained from E. coli transformed with an expression vector pENPL carrying a Neq L gene. The construction of the expression vector pENPL was achieved by cloning the Neq L gene carried by the expression vector pENPLX previously reported (Choi J. J. et al., 2006, J. Mol. Biol. 356: 1093-106; Korean Patent No. 10-0793007 and U.S. Pat. No. 7,749,732) to the same restriction enzyme site of pET-20b(+), a smaller vector, as follows. The recombinant vector pENPLX, constructed by cloning an Neq L gene to pET-22b(+) was digested with NdeI/XhoI and separated on agarose by electrophoresis, after which an Neq L gene was isolated using a gel extraction kit (Qiagen GmbH, Germany) and cloned to the same restriction enzyme site of pET-20b(+), a smaller vector, to construct a new recombinant expression vector, designated pENPL (refer to FIG. 1A). pENPL was transformed into Escherichia coli BL21-CodonPlus(DE3)-RIL (Stratagene, USA) by electroporation. The E. coli harboring the Neq L-carrying expression vector was named “Escherichia coli BL21-CodonPlus(DE3)-RIL/pENPL” and deposited in the Korean Culture Center of Microorganisms on Sep. 30, 2010 under accession No. KCCM11105P.

The Neq S protein used in the present invention was obtained from the E. coli (named “Escherichia coli BL21-CodonPlus(DE3)-RIL/pENPS”) which harbors an Neq S gene-carrying expression vector (refer to FIG. 1B) (Choi J. J. et al., 2006, J. Mol. Biol. 356: 1093-106; Korean Patent No. 10-0793007 and U.S. Pat. No. 7,749,732). The E. coli was deposited in the Korean Culture Center of Microorganisms on Sep. 30, 2010 under accession No. KCCM11104P.

FIG. 1C shows amino acid sequences of 98 and 30 amino acids of the mini inteins which correspond respectively to the carboxyl terminal part of the Neq L fragment and the amino terminal part of the Neq S fragment, both playing an important role in the protein trans-splicing-mediated hot-start PCR.

The Neq L fragment and the Neq S fragment were expressed and purified as follows. E. coli BL21-CodonPlus(DE3)-RIL strains which respectively harbored the recombinant plasmid pENPL carrying an Neq L gene and the recombinant plasmid pENPS carrying an Neq S gene were inoculated into 500 mL of respective LB media containing 100 μg/mL ampicillin and grown at 37° C. to an optical density of 0.6 at 600 nm, followed by the induction of the cloned genes in the presence of 0.2 mM IPTG at 37° C. for 6 hours. The cells were harvested by centrifugation, suspended in buffer A (20 mM Tris-HCl (pH 7.4)/50 mM KCl) containing 1 mM phenylmethylsulfonyl fluoride (hereinafter referred to as “PMSF”), a protease inhibitor, and disrupted by sonication, followed by centrifugation to separate supernatant and pellet. Herein, the purification of the Neq L fragment and the Neq S fragment was carried out using a modification of the previously reported procedure (Choi J. J. et al., 2006, J. Mol. Biol. 356: 1093-106; Korean Patent No. 10-0793007 and U.S. Pat. No. 7,749,732).

In this regard, the purification process of the Neq L fragment further comprised thermal treatment at 80° C. for 30 min, compared to the previous process, so as to effectively improve the purity. To purify the Neq L fragment, the sonicated extract was thermally treated at 80° C. for 30 min. For reference, no thermal treatment was carried out in the previous method lest the intein region should be excised from the Neq L fragment. After centrifugation, the supernatant was dialyzed against buffer B (20 mM Tris-HCl (pH 7.4)/500 mM NaCl) and loaded into a HiTrap™ Chelating HP column (Amersham Biosciences AB, Sweden), an affinity column for purifying His₆ (His-Tag)-labeled proteins, to remove most non-targeted proteins. Subsequently, fractions containing the Neq L fragment were collected and dialyzed against buffer A. The dialysate was loaded onto a UNO™ Q column (Bio-Rad Laboratories Inc., U.S.A.), an anion exchange column, to completely purify the Neq L fragment which was then dialyzed against buffer A containing 50% glycerol and stored at −20° C. The purified Neq L fragment was identified to have a molecular weight of 79,000 Da as measured by denaturation gel electrophoresis, which coincides well with the molecular weight calculated from the amino acid sequence, 79,864 Da (Choi J. J. et al., 2006, J. Mol. Biol. 356: 1093-106; Korean Patent No. 10-0793007 and U.S. Pat. No. 7,749,732).

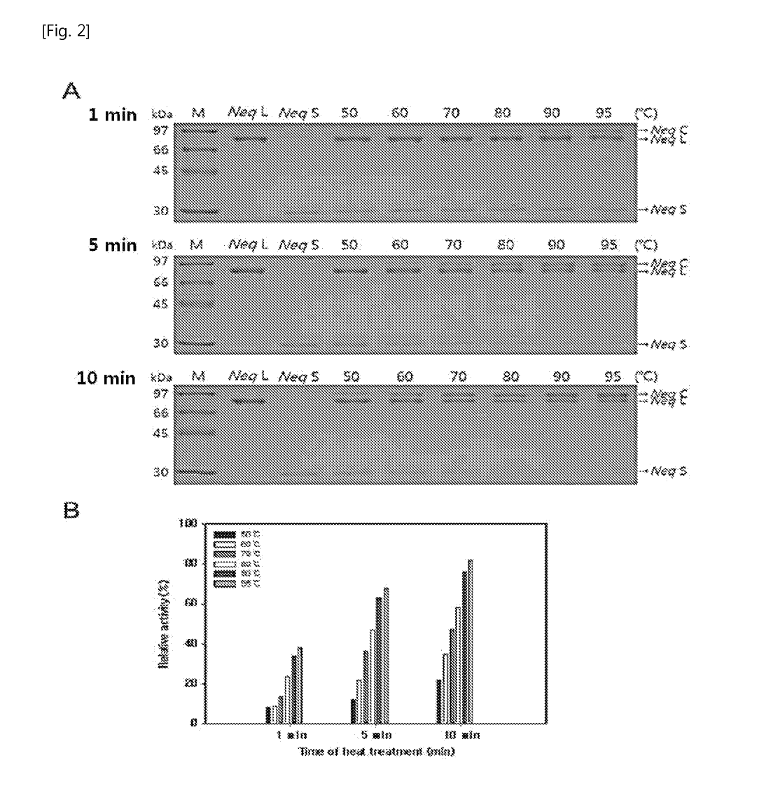

Because the Neq S fragment was expressed as an insoluble protein, it was purified in the presence of urea. Previous purification and dialysis methods were modified. The cells in which the expression of Neq S fragment was induced by IPTG were sufficiently disrupted and centrifuged at 4° C. (1851×g, 20 min) to obtain a pellet. A pre-treatment process, absent in the previous method, was carried out in which the pellet was sufficiently suspended in buffer A containing 0.2% Triton X-100 by vortexing to remove membrane proteins of E. coli from the target insoluble protein, and centrifuged in the same manner to obtain a pellet. This pre-treatment process was repeated once more to remarkably increase the purity, compared to the previous purification method. This pellet was suspended in buffer C (20 mM Tris-HCl (pH 7.4)/8 M urea) containing 1.5 M ammonium sulfate and loaded onto HiTrap™ Phenyl FF column (Amersham Biosciences AB, Sweden), a hydrophobic interaction column for purifying insoluble proteins, followed by elution using a downward linear gradient from 1.5 M to 0 M ammonium sulfate. Fractions containing the Neq S fragment were collected in a dialysis bag. The protein was refolded with the concomitant gradient removal of urea therefrom by gradient addition of buffer A containing 40% glycerol in a container of 8 M urea (Kohyama K. et al., 2010, J. Biochem. 147: 427-431) and the resulting dialysate was stored at −20° C. When the fractions were directly dialyzed against buffer A according to a previous process, as much as 90% of the proteins were lost as a precipitate. In contrast, the gradient removal process produces no precipitates and can recover almost 100% of the proteins. The molecular weight of the purified Neq S fragment was found to be 30,500 Da as measured by denaturation gel electrophoresis, which coincides well with the calculated molecular weight from the amino acid sequence, 29,538 Da (FIG. 2A).

The purified proteins Neq L and Neq S were quantitatively analyzed using the Bradford protein assay (Bradford M. M, 1976, Anal. Biochem. 72: 248-254).

### Example 2

### Effect of Temperature and Reaction Time on Protein Trans-Splicing of Neq L and Neq S Fragment

To examine the effect of temperature on protein trans-splicing, each of the Neq L fragment and the Neq S fragment, purified in Example 1, was added in an amount of 20 pmoles to a trans-splicing reaction solution (20 mM Tris-HCl (pH 8.0), 50 mM NaCl) and incubated in a temperature range of from 50 to 95° C. for 1, 5 and 10 min. Analysis by denaturation gel electrophoresis indicated that the amounts of the purified Neq L and Neq S fragments were decreased while the amounts of the protein trans-splicing products Neq C (Neq DNA polymerase) and Ext-N (intein-excised Neq L fragment) were increased (FIG. 2A). The protein trans-splicing occurred only at 60° C. or higher and was maximized at 95° C. (refer to FIG. 2A). Also, the protein trans-splicing advanced with time. FIG. 1A shows results of analyzing the relationship between protein trans-splicing of the purified Neq L and Neq S fragments with reaction temperature and time. In FIG. 1A, 20 pmoles of the purified Neq L fragment, 20 pmoles of the purified Neq S fragment and a low-molecular weight protein marker were loaded on lanes L, S and M, respectively.

FIG. 2B shows the activity of DNA polymerase after the protein trans-splicing of Neq L and Neq S fragments was induced at each temperatures for the reaction times. The activity of DNA polymerase was measured according to the previous report (Choi, J. J. et al., 2006, J. Mol. Biol. 356, 1093-1106). A reaction mixture (50 μL) comprising the purified protein, 1 μg of activated activated calf thymus DNA, 20 mM Tris-HCl (pH 8.0), 1.5 mM MgCl₂, 50 mM KCl, 100 μM deoxyadenosine 5′-triphosphate (hereinafter referred to as ‘dATP’), 100 μM deoxycytidine 5′-triphosphate (hereinafter referred to as ‘dCTP’), 100 μM deoxyguanosine 5′-triphosphate (hereinafter referred to as ‘dGTP’), 10 μM dTTP and 0.25 μCi [methyl-³H]thymidine 5′-triphosphate was incubated at 75° C. for 10 min, rapidly quenched on ice and added dropwise to a DE81 filter paper disc (23 mm, Whatman Co., U. K.). After the reaction-smeared DE81 filter paper disc was dried at 65° C., washed in 0.5 M sodium phosphate buffer (pH 7.0) for 10 min and then in 70% ethanol for 5 min, and dried again at 65° C. The radioactivity incorporated into the DE81 filter paper disc was measured using an LS6500 scintillation counter (Beckman Co., U. K.) to determine DNA polymerase activity. While the activity of 20 pmoles of Neq P (DNA polymerase expressed as a polypeptide from a recombinant gene in which an Neq L gene lacking an intein gene is ligated to the extein gene of Neq S) was set to be 100%, the activity of DNA polymerase of the solution after the protein trans-splicing of the Neq L and Neq S fragments was measured and the results are shown in FIG. 2B. The activity of DNA polymerase of the solution was very low after the protein trans-splicing of Neq L and Neq S at 60° C., but was maximized at 95° C. (refer to FIG. 2B). These results coincided well with the denaturation gel electrophoresis results of FIG. 2A.

### Example 3

### Effect of Number of PCR Cycles on Protein Trans-Splicing of Neq L and Neq S Fragments

To examine the effect of PCR conditions on protein trans-splicing in a PCR instrument, each of the Neq L fragment and the Neq S fragment, purified in Example 1, was added in an amount of 20 pmoles to a reaction solution (20 mM Tris-HCl (pH 8.0), 50 mM NaCl), and subjected to 1, 2, 3, 4, 5, 10, 20 and 30 PCR cycles of 94° C. for 20 sec, 63° C. for 20 sec, and 72° C. for 20 sec. The mixture was allowed to undergo predenaturation at 95° C. for 0, 1 or 3 min.

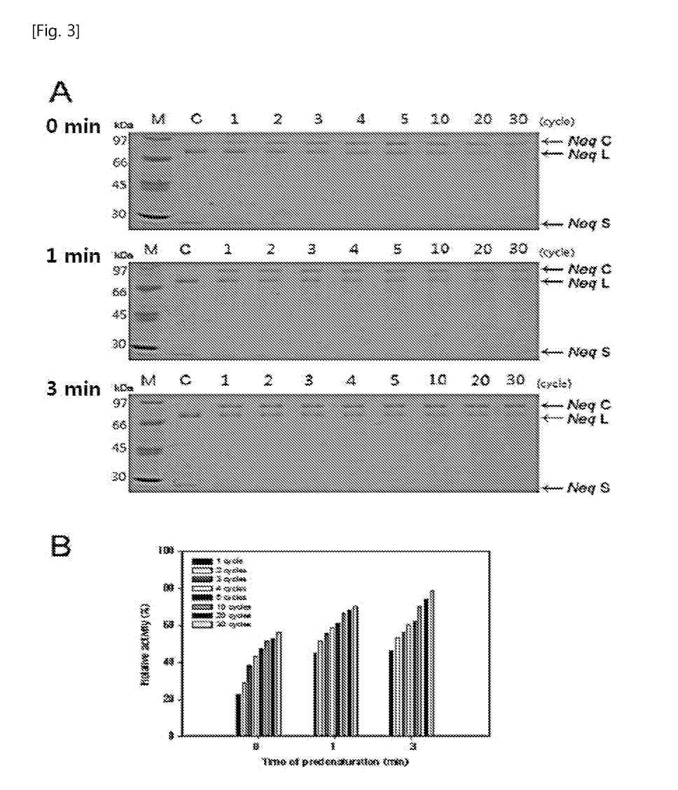

As a result, the amounts of the purified Neq L and Neq S fragments were decreased while the amounts of the protein trans-splicing products Neq C (Neq DNA polymerase) and Ext-N (intein-spliced Neq L fragment) were increased. The protein trans-splicing advanced with an increase in the number of PCR cycles (refer to FIG. 3A). With a small number of PCR cycles, the protein trans-splicing was observed to depend on the period of time of the pre-denaturation. In contrast, for a large number of PCR cycles, the protein trans-splicing proceeded well irrespective of the period of time of the pre-denaturation (refer to FIG. 3A). FIG. 3B shows the activity of DNA polymerase according to the number of PCR cycles, as measured in the same manner as in Example 2. While the activity of 20 pmoles of Neq P was set to be 100%, the activity of DNA polymerase of the solution was measured. The results were similar to those of FIG. 2A (refer to FIG. 3B).

### Example 4

### Effect of Concentration of Equimolar Neq L and Neq S Fragment Mixture on PCR

As illustrated in Examples 2 and 3, when equal molar concentrations the Neq L fragment and the Neq S fragment were used, they were well processed by protein trans-splicing. In those cases, the amounts of the proteins exceeded the protein amounts typically used for PCR in order to visualize the results of the protein trans-splicing. Because most PCR reactions do not proceed when the amount of DNA polymerase is excessive or present in a small amount, it is important to optimize the amount of DNA polymerase. Like other enzymes, Neq DNA polymerase does not amplify DNA when it is used in an excessive amount. Therefore, the mixture of equimolar amounts of Neq L and Neq S fragments employed for the protein trans-splicing was diluted before application to PCR experiments.

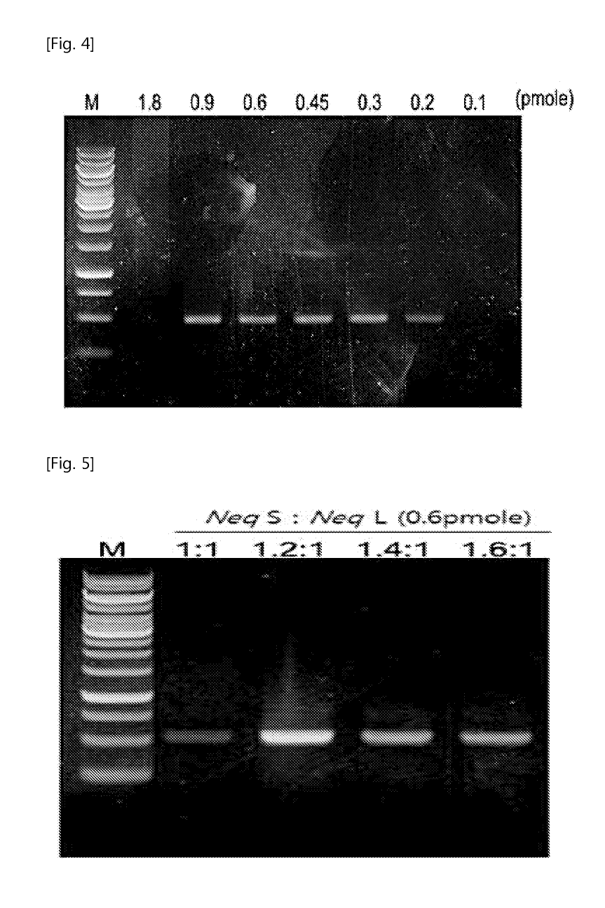

In this context, 20 μl of the PCR reaction mixture in which a mixture of equimolar Neq L and Neq S fragments were used at various concentrations was subjected to PCR amplification. In order to compare the amplification efficiency according to the amount of the Neq L and Neq S fragments, a 500 bp lambda DNA was amplified by PCR using primers (forward primer Lambda-1F and reverse primer Lambda-1R). The base sequences of the primers Lambda-1F and Lambda-1R were 5′-AATAACGTCGGCAACTTTGG-3′ (SEQ. ID NO. 6) and 5′-GTTACGCCACCAGTCATCCT-3′ (SEQ. ID NO. 7), respectively. In 20 μl of the PCR reaction mixture, equimolar Neq L and Neq S fragments were present at a total concentration of 0.2 to 1.8 pmol, together with 10 pmole of each of the primers, 250 μM dNTP, and 1 ng of lambda DNA. After DNA pre-denaturation at 94° C. for 2 min, PCR was performed with 30 cycles of denaturation at 95° C. for 20 sec, annealing at 63° C. for 20 sec and DNA extension at 72° C. for 30 sec. The PCR products thus obtained from the different amounts of equimolar Neq L and Neq S fragments were separated by agarose gel electrophoresis and the results are shown in FIG. 4. In FIG. 4, 1 kb marker was run on lane M, Neq L/Neq S (1.8 pmole) on lane 1, Neq L/Neq S (0.9 pmole) on lane 2, Neq L/Neq S (0.6 pmole) on lane 3, Neq L/Neq S (0.45 pmole) on lane 4, Neq L/Neq S (0.3 pmole) on lane 5, Neq L/Neq S (0.2 pmole) on lane 6, and Neq L/Neq S (0.1 pmole) on lane 7. As can be seen in FIG. 4, the DNA was amplified at a concentration of the Neq L and Neq S fragment in 20 μL of the PCR reaction mixture, but was not amplified at a concentration less than 0.1 pmole or more than 0.9 pmoles.

### Example 5

### Effect of Molar Ratio Between Neq L and Neq S on PCR Amplification

As elucidated in Examples 2 and 3, the protein trans-splicing of the Neq L fragment and the Neq S fragment clearly occurred when the molar amounts of the fragments were equal. In Example 4, DNA amplification was found to proceed effectively when the total concentration of both Neq L and Neq S in 20 μL of a PCR reaction mixture was on the order of 0.2˜0.9 pmoles. Thus, while an equimolar mixture of Neq L and Neq S was used in an amount of 0.2 pmoles as a fundamental set, PCR amplification was analyzed for different molar ratios between the Neq L and the Neq S fragment. In order to compare the amplification efficiency according to the molar ratio of the Neq L and Neq S fragments, a 500 bp lambda DNA was amplified by PCR using primers (forward primer Lambda-1F and reverse primer Lambda-1R). The base sequences of the primers Lambda-1F and Lambda-1R were 5′-AATAACGTCGGCAACTTTGG-3′ (SEQ. ID NO. 6) and 5′-GTTACGCCACCAGTCATCCT-3′ (SEQ. ID NO. 7), respectively. In 20 μl of the PCR reaction mixture, 0.6 pmoles of the Neq L fragment and 0.6-0.96 pmoles of the Neq S fragment were contained, together with 10 pmole of each primers, 250 μM dNTP, and 1 ng of lambda DNA. After DNA pre-denaturation at 95° C. for 2 min, PCR was performed with 30 cycles of denaturation at 94° C. for 20 sec, annealing at 63° C. for 20 sec and DNA extension at 72° C. for 30 sec. The PCR products thus obtained at different molar ratios between Neq L and Neq S fragments were separated by agarose gel electrophoresis and the results are shown in FIG. 5. In FIG. 5, 1 kb marker was run on lane M, Neq L 0.6 pmole/Neq S 0.6 pmole (1:1) on lane, Neq L 0.6 pmole/Neq S 0.72 pmole (1:1.2) on lane 2, Neq L 0.6 pmole/Neq S 0.84 pmole (1:1.4) on lane 3, and Neq L 0.6 pmole/Neq S 0.96 pmole (1:1.6) on lane 4. As can be seen in FIG. 5, the DNA was amplified over a molar ratio range of Neq L to Neq S from 1:1 to 1:1.6 with a peak at a molar ratio of 1:1.2.

### Example 6

### Analysis of PCR Efficiency of DNA Polymerases

As elucidated in Examples 4 and 5, the combinational use of 0.6 pmoles of Neq L and 0.72-0.84 pmoles of Neq S in 20 μL of a PCR reaction mixture was found to be effective for PCR. Herein, the DNA polymerase produced from a combination of Neq L and Neq S was designated Neq HS DNA, which is discriminated from conventional Neq DNA polymerase.

In order to analyze DNA polymerases for accuracy and efficiency in DNA amplification, a 653 bp-long β-actin gene and a 1581 bp-long hemoglobin gene on the human chromosome were selected as targets to be amplified. For the amplification, Neq HS DNA polymerase (a combination of 0.6 pmoles of Neq L and 0.72 pmoles of Neq S), 1 U Taq DNA polymerase and 1 U Pfu DNA polymerase was used.

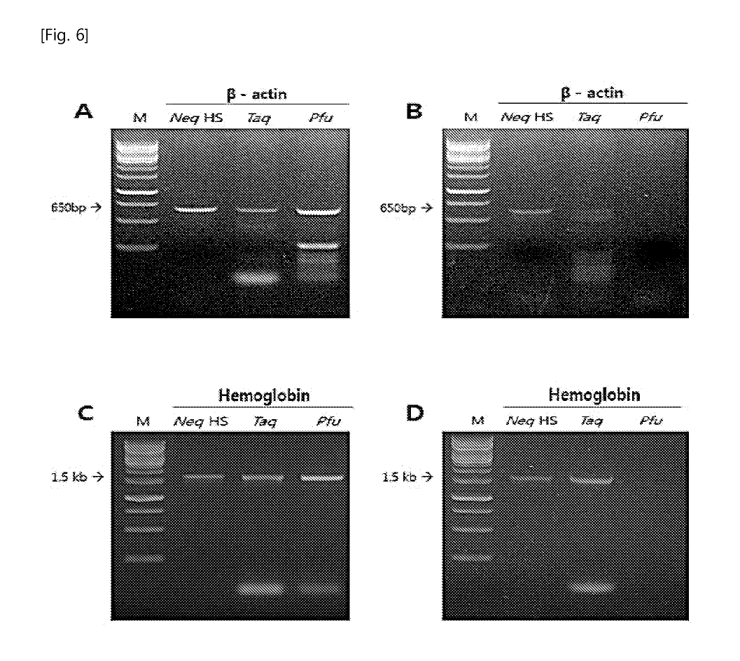

In addition to the DNA polymerase, the PCR reaction mixture contained a pair of primers targeting the 653 bp-long fragment of the β-actin gene on the human genome (5 pmoles of forward primer: 5′-AGAGATGGCCACGGCTGCTT-3′ (SEQ. ID NO. 8); reverse primer: 5′-ATTGCGGTGGACGATGGAG-3′(SEQ. ID NO. 9)), 10 ng of human genomic DNA, and 250 μM dNTPs (dATP, dCTP, dGTP, dTTP). PCR started with pre-denaturation at 95° C. for 3 min and proceeded with 40 cycles of denaturation at 94° C. for 20 sec, annealing at 60° C. for 20 sec and extension at 72° C. for 40 sec, followed by extension at 72° C. for 5 min. The PCR products thus obtained were separated by agarose gel electrophoresis (FIG. 6A). As can be seen in FIG. 6A, Neq HS DNA polymerase was observed to amplify the target DNA at higher specificity than did the other DNA polymerases. PCR was also performed to amplify the 653 bp-long fragment of β-actin gene in the same manner, with the exception that dUTP was used instead of dTTP. The PCR products were identified by electrophoresis (FIG. 6B), demonstrating that Neq HS DNA polymerase more specifically amplified the target DNA than did the other DNA polymerases. Particularly, Pfu DNA polymerase did not amplify the target DNA at all because it cannot utilize dUTP.

A 1581 bp hemoglobin gene on the human genome was also selected as a PCR amplification target. For use in the amplification, a PCR reaction mixture contained a pair of primers targeting the hemoglobin gene (5 pmoles of forward primer: 5′-ACATTTGCTTCTGACACAACTG-3′ (SEQ. ID NO. 10); and reverse primer: 5′-AGGCAGAATCCAGATGCTCAA-3′ (SEQ. ID NO. 11)), 10 ng of human genomic DNA, and 250 μM dNTPs (dATP, dCTP, dGTP, dTTP). The polymerases were used in the same amounts as described above. PCR started with pre-denaturation at 95° C. for 3 min and proceeded with 40 cycles of denaturation at 94° C. for 20 sec, annealing at 60° C. for 20 sec and extension at 72° C. for 40 sec, followed by extension at 72° C. for 5 min. The PCR products thus obtained were separated by agarose gel electrophoresis (FIG. 6C). As can be seen in FIG. 6C, Neq HS DNA polymerase was observed to amplify the target DNA at higher specificity than did the other DNA polymerases. PCR was also performed to amplify the 1581 bp-long fragment of hemoglobin gene in the same manner, with the exception that dUTP was used instead of dTTP. The PCR products were identified by electrophoresis (FIG. 6D), demonstrating that Neq HS DNA polymerase more specifically amplified the target DNA than did the other DNA polymerases. As explained in FIG. 5B, Pfu DNA polymerase did not amplify the target DNA at all because it cannot utilize dUTP. Consequently, the accuracy of the DNA amplification of Neq HS DNA polymerase is superior to that of other DNA polymerases.

Taken together, the data obtained above indicate that the hot-start PCR method of the present invention can more accurately amplify just the DNA targets of interest without non-specific amplification, compared to Taq DNA polymerase and Pfu DNA polymerase and that when dUTP is used, the hot-start PCR method based on protein trans-splicing in accordance with the present invention can amplify DNA targets of interest more selectively, compared to other DNA polymerases.

### INDUSTRIAL APPLICABILITY

In accordance with the present invention, a novel hot-start PCR method based on the protein trans-splicing of Neq L and Neq S fragments of Nanoarchaeum equitans DNA polymerase is provided. The hot-start PCR method guarantees the amplification of target DNA with higher accuracy, compared to PCR methods using conventional DNA polymerases. Particularly in the presence of dUTP, the DNA polymerase of the present invention exhibits more specific amplification than did Taq DNA polymerase, indicating that the hot-start PCR method of the present invention can utilize dUTP and UDG in PCR for preventing carry-over contamination at high efficiency. Therefore, the present invention may be more effective than Taq DNA polymerase in applications in various fields including genetic engineering and molecular biology experiments, clinical diagnosis, forensic studies, etc.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

Deposition No.

Depository Authority Korean Culture Center of Microorganisms (outside)

Accession No.: KCCM11104P

Deposition Date Sep. 30, 2010

Depository Authority: Korean Culture Center of Microorganisms (outside)

Accession No.: KCCM11105P

Deposition Date Sep. 30, 2010

What is claimed is:

1. A hot-start PCR method, based on protein trans-splicing of intein-inserted large (Neq L) and small (Neq S) fragments of Neq DNA polymerase, comprising:
preparing a PCR reaction mixture containing a sample DNA and primers;
adding the Neq L fragment and the Neq S fragment together to the PCR reaction mixture, said Neq L fragment consisting of an amino acid sequence of SEQ ID NO: 2, with an intein amino acid sequence stretching from position 579 to 676 therein, said Neq S fragment consisting of an amino acid sequence of SEQ ID NO: 4 with an intein amino acid sequence stretching from position 1 to 30 therein;
inducing the Neq L fragment and the Neq S fragment to undergo a protein trans-splicing process to form a polypeptide (SEQ ID NO: 5) exhibiting Neq DNA polymerase activity; and
performing a certain number of cycles of DNA denaturation, primer annealing and DNA extension.

2. The hot-start PCR method of claim 1, wherein the polypeptide exhibiting Neq DNA polymerase activity is produced after the trans-splicing of both the Neq L fragment and the Neq S fragment at 60˜95° C. for 2˜9 min.

3. The hot-start PCR method of claim 1, wherein the PCR reaction mixture contains each of the Neq L fragment and the Neq S fragment at a concentration of 0.2 to 0.9 pmol.

4. The hot-start PCR method of claim 1, wherein the PCR reaction mixture contains the Neq L and the Neq S fragments at a molar ratio of from 1:1 to 1:1.6.

5. The hot-start PCR method of claim 1, wherein the PCR is performed in a presence of dUTP (2′-deoxyuridine 5′-triphosphate).

6. A hot-start PCR method, based on protein trans-splicing of intein-inserted large (Neq L) and small (Neq S) fragments of Neq DNA polymerase, comprising:
preparing a PCR reaction mixture containing a sample DNA and primers;
adding a Neq L fragment and a Neq S fragment together to the PCR reaction mixture, said Neq L fragment comprising an amino acid sequence having about 95% to less than 100% similarity to SEQ ID NO:2, with an intein amino acid sequence stretching from position 579 to 676 of SEQ ID NO:2, said Neq S fragment comprising an amino acid sequence having about 95% to less than 100% similarity to SEQ ID NO: 4 with an intein amino acid sequence stretching from position 1 to 30 of SEQ ID NO: 4;
inducing the Neq L fragment and the Neq S fragment to undergo a protein trans-splicing process to form a polypeptide comprising an amino acid sequence having about 95% to less than 100% similarity to SEQ ID NO: 5 exhibiting Neq DNA polymerase activity; and
performing a certain number of cycles of DNA denaturation, primer annealing and DNA extension.

7. The hot-start PCR method according to claim 6, wherein said Neq L fragment comprises an amino acid sequence having about 96% to less than 100% similarity SEQ ID NO:2.

8. The hot-start PCR method according to claim 6, wherein said Neq L fragment comprises an amino acid sequence having about 97% to less than 100% similarity SEQ ID NO:2.

9. The hot-start PCR method according to claim 6, wherein said Neq L fragment comprises an amino acid sequence having about 98% to less than 100% similarity SEQ ID NO:2.

10. The hot-start PCR method according to claim 6, wherein said Neq L fragment comprises an amino acid sequence having about 99% to less than 100% similarity SEQ ID NO:2.

11. The hot-start PCR method according to claim 6, wherein said Neq S fragment comprises an amino acid sequence having about 96% to less than 100% similarity to SEQ ID NO: 4.

12. The hot-start PCR method according to claim 6, wherein said Neq S fragment comprises an amino acid sequence having about 97% to less than 100% similarity to SEQ ID NO: 4.

13. The hot-start PCR method according to claim 6, wherein said Neq S fragment comprises an amino acid sequence having about 98% to less than 100% similarity to SEQ ID NO: 4.

14. The hot-start PCR method according to claim 6, wherein said Neq S fragment comprises an amino acid sequence having about 99% to less than 100% similarity to SEQ ID NO: 4.

15. The hot-start PCR method according to claim 6, wherein said formed polypeptide comprises an amino acid sequence having about 96% to less than 100% similarity to SEQ ID NO: 5.

16. The hot-start PCR method according to claim 6, wherein said formed polypeptide comprises an amino acid sequence having about 97% to less than 100% similarity to SEQ ID NO: 5.

17. The hot-start PCR method according to claim 6, wherein said formed polypeptide comprises an amino acid sequence having about 98% to less than 100% similarity to SEQ ID NO: 5.

18. The hot-start PCR method according to claim 6, wherein said formed polypeptide comprises an amino acid sequence having about 99% to less than 100% similarity to SEQ ID NO: 5.
